(19) United States
(12) Patent Application Publication
(10) Pub. No.: US 2010/0063349 A1
(43) Pub. Date: Mar. 11, 2010

(54) APPARATUS AND ELECTRONIC DEVICE FOR INDUCING BRAIN WAVE

(75) Inventors: TAO HE, Shenzhen City (CN); YONG CHEN, Shenzhen City (CN); CHING-KUEI CHIEN, Tu-Cheng (TW)

Correspondence Address: PCE INDUSTRY, INC.;ATT. Steven Reiss, 288 SOUTH MAYO AVENUE, CITY OF INDUSTRY, CA, 91789, US

(73) Assignees: HON FU JIN PRECISION INDUSTRY (Shenzhen) CO., LTD., Shenzhen City (TW); HON HAI PRECISION INDUSTRY CO., LTD, Taipei Hsien (TW)

(21) Appl. No.: 12/551,424

(22) Filed: Aug. 31, 2009

(30) Foreign Application Priority Data
Sep. 5, 2008 (CN) 200810304393.X

(51) Int. Cl. A61M 21/00

(52) U.S. Cl. 600/27

(57) ABSTRACT

A brain wave inducing apparatus includes a signal generator and at least one sensor electrically coupled to the generator. The signal generator generates electrical signals with frequencies in a predetermined range. The at least one sensor is electrically coupled to the signal generator and detachably attached to a human body. The at least one sensor generates vibration at a frequency in the predetermined range in response to the electrical signals. The vibration is transferred to a the human body to induce the human brain to generate brain waves with the frequencies in the predetermined range.

### BACKGROUND

1. Technical Field

The present disclosure relates to electronic devices, and particularly to an electronic device for inducing a human brain to generate a desired brain wave.

2. Description of Related Art

It is well known that the human brain generates periodic electrical signals, commonly referred to as “brain wave”. The brain waves are classified into beta, alpha, delta, and theta brain waves according to their frequencies.

In general, the beta brain waves, with frequencies of 14 hertz (Hz) or higher, are generated in states of increased brain activity, or when working under strain. The alpha brain waves, with frequencies of 8 to 14 Hz, are generated under stable conditions of a normal person, such as during work, simple learning, meditation, etc. The theta brain waves, with frequencies of 4 to 7 Hz, are generated during light sleep. The delta brain waves, with frequencies of lower than 4 Hz, are generated during deep sleep. A person with high work efficiency can produce more things in less time. In addition, work efficiency is closely linked with better quality of work. Thus if a person brain generates alpha waves for extended periods, that person work efficiency will improve.

Therefore, an apparatus and an electronic device for inducing the human brain to generate the alpha brain wave are desired.

Other advantages and novel features will become more apparent from the following detailed description of exemplary embodiments when taken in conjunction with the accompanying drawings.

### BRIEF DESCRIPTION OF THE DRAWINGS

Many aspects of the embodiments can be better understood with reference to the following drawings. The components in the drawings are not necessarily drawn to scale, the emphasis instead being placed upon clearly illustrating the principles of the present embodiments. Moreover, in the drawings, like reference numerals designate corresponding parts throughout the several views.

FIG. 1 is a functional diagram of a brain wave inducing apparatus in accordance with a first exemplary embodiment, wherein the brain wave inducing apparatus includes a signal generator.

FIG. 2 is a detailed functional diagram of the signal generator of FIG. 1 in accordance with the exemplary embodiment.

FIG. 3 is a functional diagram of an electronic device using the brain wave inducing apparatus of FIG. 1 in accordance with a second exemplary embodiment.

### DETAILED DESCRIPTION

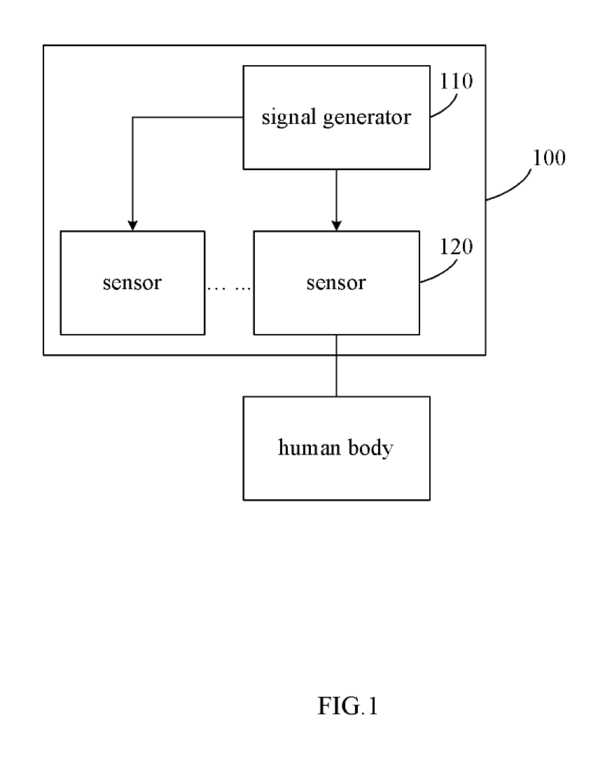

Referring to FIG. 1, the present disclosure provides a brain wave inducing apparatus 100 for inducing, by applying vibration to a human body of a person, the brain wave of the person to be in a desired frequency range. In one embodiment, the vibrating frequencies are in a range of 8-14 hertz (HZ), such that the desired brain waves induced are alpha brain waves.

The brain wave inducing apparatus 100 includes a signal generator 110 and a plurality of sensors 120. The signal generator 110 is configured to generate electrical signals with frequencies of 8-14 HZ. The electrical signals may include regular sinusoidal waves. The plurality of sensors 120 are configured to vibrate when receiving the electrical signals. The sensors 120 may be piezoelectric sensors that deform at a rate according to the electrical signals. As a result, the sensors 120 will vibrate at frequencies of the electrical signals if the frequencies of the electrical signals are between 8 HZ and 14 HZ. For example, the frequencies of the electrical signals may be adjusted to be in the range of 8-14 HZ, thus, the plurality of sensors 120 deform at frequencies in the range of 8-14 HZ. In such a manner, the plurality of sensors 120 vibrates at frequencies in the range of 8-14 HZ. If the plurality of sensors 120 are placed in touch with the human body, the vibration generated by the plurality of sensors 120 will be transferred to the human body, such that the brain is stimulated to generate the alpha brain waves. In other words, the brain wave inducing apparatus 100 applies vibration to the human body by placing the sensors 120 on the skin. How the signal generator 110 generates the electrical signals and how the plurality of sensors 120 apply vibration to the human body will be described as follows.

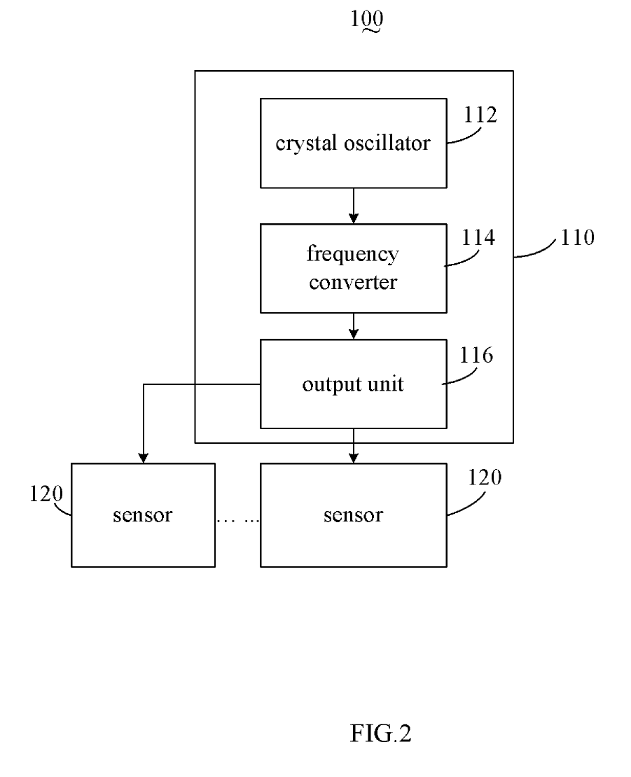

Referring to FIG. 2, the signal generator 110 includes a crystal oscillator 112, a frequency convertor 114, and an output unit 116. The crystal oscillator 112 is configured for generating clock signals. The frequency convertor 114 is configured for converting frequencies of the clock signals into 8-14 HZ, in other words, the electrical signals are generated. The frequency convertor 114 is further configured to output the electrical signals to the plurality of sensors 120 via the output unit 116. The plurality of sensors 120 are electrically coupled to the signal generator 110, and can be, as needed, coupled to a human body. For example, in one embodiment, the plurality of sensors 120 are placed on the human body, thus the vibration of the plurality of sensors 120 can be directly transferred to the human body. In another embodiment, the plurality of sensors 120 attached to the human body via an object. For example, the object may be a part of a chair back, or a part of pillow. When a person sits on the chair or lie on the pillow, the plurality of sensor 120 comes in contact to the human body. As such, the vibration generated by the sensors 120 can be indirectly transferred to the human body.

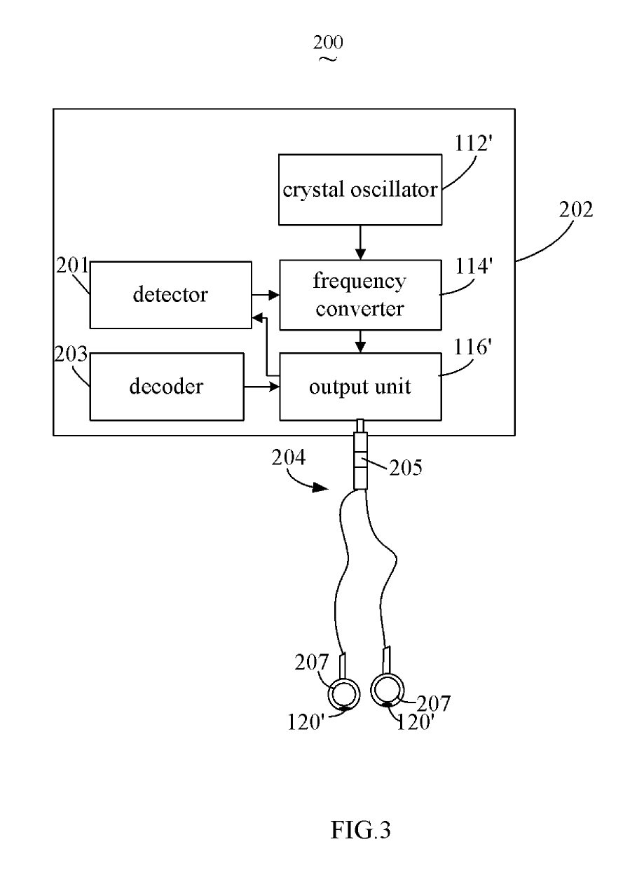

The brain wave inducing apparatus 100 of FIG. 1 may be an independent device as described above. Referring to FIG. 3, the brain wave inducing apparatus 100 may also be a functional module for an electronic device 200 which uses a crystal oscillator to generate real time clock signals. The electronic device 200 may be a mobile phone, a personal digital assistant (PDA) and so on. For example, the electronic device 200 includes a main body 202 and an earphone 204 which are detachably and electrically coupled to the main body 202. The main body 202 includes a crystal oscillator 112′, a frequency convertor 114′ and an output unit 116′, a detector 201, and a decoder 203. It is understood the main body 202 may include other functional modules (not shown) such as a processor or a memory. The crystal oscillator 112′, the frequency convertor 114′ and the output unit 116′ form the brain wave inducing apparatus 100 of FIG. 1. The crystal oscillator 112′ is configured for providing clock signals to the frequency convertor 114′ and other functional modules. The detector 201 is configured for detecting whether the earphone 204 is coupled to the main body 202 or not, and generates a start signal when the earphone 204 is coupled to the main body 202. The frequency convertor 114′ is configured for converting frequencies of the clock signals into 8-14 HZ when receiving the start signal. In other words, the frequency convertor 114′ generates the electrical signals when the earphone 204 is coupled to main body 202. The output unit 116′ may also be an audio interface of the electronic device 200 which couples the earphone 204 to the main body 202. The decoder 203 is configured for decoding audio files stored in a medium such as an optical disc and/or a flash memory card. It is understood the audio files may be retrieved from external devices or a network.

The earphone 204 includes a plug 205, two earpieces 207, and a plurality of sensors 120′. In this embodiment, the plurality of sensors 120′ includes two sensors 120′ which are similar with the sensors 120. The plug 205 is inserted into the output unit 116 to electrically couple the earphone 204 to the main body 202. As such, the detector 201 may detect whether the plug 205 has been inserted into the output unit 116 or not. The two sensors 120′ are attached to the earpieces 207 and are positioned on the periphery of the earpieces 207 respectively. As such, if the earpieces 207 are plugged into one's ears (not shown), the sensors 120′ are in contact with the inner ears. Furthermore, music and other audio programs can also be reproduced from the main body 202 with the earphone 204. As such, alpha brain waves can be induced but music can also be provided to relax.

As described above, the brain wave inducing apparatus 100 and the electronic device 200 generates the vibration whose frequency is in the range of 8-14 HZ. The vibration is then applied to human body to simulate the alpha brain waves. Therefore, the person can focus attention on his work to improve woke efficiency

It is believed that the present embodiments and their advantages will be understood from the foregoing description, and it will be apparent that various changes may be made thereto without departing from the spirit and scope of the invention or sacrificing all of its material advantages, the examples hereinbefore described merely being preferred or exemplary embodiments of the invention.

What is claimed is:

1. A brain wave inducing apparatus, comprising:
a signal generator configured to generate electrical signals with frequencies in a predetermined range, and
at least one sensor electrically coupled to the signal generator and detachably attached to a human body, the at least one sensor configured to generate vibration at a frequency in the predetermined range according to the electrical signals, and to transfer the vibration to the human body to induce the human brain attached to the human body to generate brain waves with frequencies in the predetermined range.

2. The brain wave inducing apparatus of claim 1, wherein the predetermined range is 8-14 HZ.

3. The brain wave inducing apparatus of claim 1, wherein the signal generator comprises:
a crystal oscillator configured to generate clock signals;
a frequency convertor configured to convert frequencies of the clock signals into the predetermined range to generate the electrical signals; and
an output unit configured to transmit the electrical signals from the frequency convertor to the at least one sensor.

4. The brain wave inducing apparatus of claim 1, wherein the electrical signals comprise regular sinusoidal waves.

5. The brain wave inducing apparatus of claim 1, wherein the at least one sensor is directly in contact with the human body.

6. The brain wave inducing apparatus of claim 1, wherein the at least one sensor is indirectly in contact with the human body via an object.

7. The brain wave inducing apparatus of claim 1, wherein the at least one sensors are piezoelectric sensors.

8. A brain wave inducing apparatus, comprising:
a crystal oscillator configured to generate clock signals;
a frequency convertor configured to convert frequencies of the clock signals into a predetermined range to generate electrical signals;
an output unit configured to receive and transmit the electrical signals from the frequency convertor;
at least one sensor electrically coupled to the frequency convertor via the output unit and attached to a human body, the at least one sensor configured to generate vibration at a predetermined frequency in response to the electrical signals transmitted from the output unit, and transfer the vibration to the human body to induce a human brain attached to the human body to generate brain waves with substantially the same frequency of the electrical signals and the vibration.

9. The brain wave inducing apparatus of claim 8, wherein the predetermined range is 8-14 HZ.

10. The brain wave inducing apparatus of claim 8, wherein the at least one sensor is caused to deform by the electrical signals to generate the vibration.

11. The brain wave inducing apparatus of claim 8, wherein the electrical signals comprise regular sinusoidal waves.

12. The brain wave inducing apparatus of claim 8, wherein the at least one sensor is directly in contact with a part of the human body.

13. The brain wave inducing apparatus of claim 8, wherein the at least one sensor is indirectly in contact with a part of the human body via an object.

14. An electronic device, comprising:
a main body configured to generate electrical signals, the main body comprising:
a crystal oscillator configured to generate clock signals;
a frequency convertor configured to convert frequencies of the clock signals into a predetermined range to generate the electrical signals;
an output unit configured to receive and transmit the electrical signals from the frequency convertor; and
an earphone detachably and electrically coupled to the frequency convertor via the output unit, the earphone comprising an earpiece and at least one sensor attached to the earpiece, the at least one sensor generating vibration with a frequency in the predetermined range in response to the electrical signals.

15. The electronic device of claim 14, further comprising a detector, the detector configured to detect whether the earphone is coupled to the frequency convertor, and generate a start signal when the earphone is detected to be coupled to the frequency convertor, the frequency convertor starts to convert the frequencies of the electrical signals into the predetermined range in response to the start signal.

16. The electronic device of claim 14, wherein the predetermined range is 8-14 HZ.

17. The electronic device of claim 14, wherein the at least one sensor deforms at a frequency in the predetermined range caused by the electrical signals to generate the vibration.

18. The electronic device of claim 14, wherein the electrical signals comprise regular sinusoidal waves.

19. The electronic device of claim 14, wherein the at least one sensor is directly in contact with a part of the human body.

20. The electronic device of claim 14, wherein the at least one sensor is indirectly in contact with a part of the human body via an object.
